SEQUENCE LISTING (1 sequence)

SEQ ID NO: 1 - protein, 21 aa, ARTIFICIAL SEQUENCE
  HLA-G5/-G6
SKEGDGGIMSVRESRSLSEDL

(19) United States
(12) Patent Application Publication
(10) Pub. No.: US 2007/0020703 A1
(43) Pub. Date: Jan. 25, 2007

(54) Use of compositions comprising a soluble form of hla-g in the treatment of blood diseases

(75) Inventors: Catherine Menier, Maisons-Alfort (FR); Edgardo Delfino Carosella, Paris (FR); Nathalie Rouas-Freiss, Paris (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: COMMISSARIAT A L'ENERGIE ATOMIQUE, PARIS (FR)

(21) Appl. No.: 10/562,659

(22) PCT Filed: Jun. 29, 2004
(86) PCT No.: PCT/FR04/01663

§ 371 Date: Dec. 29, 2005

(30) Foreign Application Priority Data
Jun. 30, 2003 (FR) 0307871

(51) Int. Cl. G01N 33/567; A61K 38/17

(52) U.S. Cl. 435/7.21; 514/12

(57) ABSTRACT

The invention relates to the use of compositions comprising a soluble form of HLA-G in the treatment of blood and circulatory system diseases (anemias and ischaemias).

The present invention relates to the use of compositions comprising a soluble form of HLA-G, in the treatment of blood and circulatory system diseases (anemias and ischemias).

The major histocompatibility complex (MHC) antigens are divided up into several classes, class I antigens (HLA-A, HLA-B and HLA-C), which have 3 globular domains (α1, α2 and α3), and in which the a3 domain is associated with β2-microglobulin, class II antigens (HLA-DP, HLA-DQ and HLA-DR) and class III antigens (complement).

Class I antigens comprise, in addition to the abovementioned antigens, other antigens, called nonclassical class I antigens, and in particular the antigens HLA-E, HLA-F and HLA-G.

The sequence of the HLA-G gene (HLA-6.0 gene) has been described by Geraghty et al., (Proc. Natl. Acad. Sci. USA, 1987, 84, 9145-9149): it comprises 4396 base pairs and has an intron/exon organization homologous to that of the HLA-A, -B and -C genes. More specifically, this gene comprises 8 exons, 7 introns and an untranslated 3′ end; the 8 exons correspond, respectively, to: exon 1: signal sequence, exon 2: α1 extracellular domain, exon 3: α2 extracellular domain, exon 4: α3 extracellular domain, exon 5: transmembrane region, exon 6: cytoplasmic domain I, exon 7: cytoplasmic domain II (untranslated), exon 8: cytoplasmic domain III (untranslated) and 3′ untranslated region (Geraghty et al., mentioned above; Ellis et al., J. Immunol., 1990, 144, 731-735; Kirszenbaum M. et al., Oncogeny of hematopoiesis. Aplastic anemia Eds. E. Gluckman, L. Coulombel, Colloque INSERM/John Libbey Eurotext Ltd). However, the HLA-G gene differs from the other class I genes in that the in-frame translation stop codon is located at the second codon of exon 6; consequently, the cytoplasmic region of the protein encoded by this HLA-6.0 gene is shorter than the cytoplasmic regions of the HLA-A, -B and -C proteins.

These HLA-G antigens are essentially expressed by the cytotrophoblast cells of the placenta and are considered to play a role in protection of the fetus (lack of rejection by the mother). In addition, insofar as the HLA-G antigen is monomorphic, it may also be involved in placental cell growth or function (Kovats et al., Science, 1990, 248, 220-223).

Other research studies concerning this nonclassical class I antigen (Ishitani et al., Proc. Natl. Acad. Sci. USA, 1992, 89, 3947-3951) have shown that the primary transcript of the HLA-G gene can be spliced in many ways and produce at least 3 distinct mature mRNAs: the primary transcript of HLA-G provides a complete copy (G1) of 1 200 bp, a 900 bp fragment (G2) and a 600 bp fragment (G3).

The G1 transcript does not comprise exon 7 and corresponds to the sequence described by Ellis et al. (mentioned above), i.e. it encodes a protein that comprises a signal sequence, three external domains, a transmembrane region and a cytoplasmic sequence. The G2 mRNA does not comprise exon 3, i.e. it encodes a protein in which the α1 and α3 domains are directly attached; the G3 mRNA contains neither exon 3 nor exon 4, i.e. it encodes a protein in which the α1 domain and the transmembrane sequence are directly attached.

The splicing that prevails in order to obtain the HLA-G2 antigen results in the adjoining of an adenine (A) (originating from the α1 coding domain) with a sequence AC (derived from the α3 coding domain), which results in the creation of an AAC codon (asparagine) in place of the GAC codon (aspartic acid), encountered at the beginning of the sequence encoding the α3 domain in HLA-G1.

The splicing generated in order to obtain HLA-G3 does not result in the formation of a new codon in the splicing zone.

The authors of that article also analyzed the various proteins expressed: the 3 mRNAs are translated into protein in the 0.221-G cell line.

The authors of that article conclude that the HLA-G molecule has a fundamental role in protecting the fetus against a maternal immune response (induction of immune tolerance). Some of the inventors have confirmed this role: HLA-G molecules, expressed at the surface of trophoblasts, effectively protect fetal cells against lysis by maternal natural killer (NK) cells (Carosella E. D. et al., C.R. Acad. Sci., 318, 827-830; Carosella E. D. et al; Immunol. Today, 1996, 407-409).

In addition, some of the inventors have shown the existence of other spliced forms of HLA-G mRNA: the HLA-G4 transcript, which does not include exon 4; the HLA-G5 transcript, which includes intron 4, between the exons 4 and 5, thus causing a modification of the reading frame, during translation of this transcript, and in particular the appearance of a stop codon, after amino acid 21 of intron 4; the HLA-G6 transcript, which has intron 4 but has lost exon 3 (Kirszenbaum M. et al., Proc. Natl. Acad. Sci. USA, 1994, 91, 4209-4213; European application EP 0 677 582; Kirszenbaum M. et al., Human Immunol., 1995, 43, 237-241; Moreau P. et al., Human Immunol. 1995, 43, 231-236); and the HLA-G7 transcript, which includes intron 2, thus causing a modification of the reading frame, during translation of this transcript, and the appearance of a stop codon after amino acid 2 of intron 2; they have also shown that these various transcripts are expressed in several types of fetal and adult human cells, in particular in lymphocytes (Kirszenbaum M. et al., Human Immunol., 1995, mentioned above; Moreau P. et al., Human Immunol. 1995, mentioned above).

At least 7 different HLA-G mRNAs therefore exist, which potentially encode 7 HLA-G isoforms, of which 4 are membrane isoforms (HLA-G1, G2, G3 and G4) and 3 are soluble isoforms (HLA-G5, G6 and G7).

The distribution of the HLA-G antigen is restricted to immune-privileged sites, and in particular to the feto-maternal interface.

It is now clearly established that the membrane-bound HLA-G1 protein is mainly expressed in the extravillous cytotrophoblast cells, in which it protects the fetus against immune cells of maternal origin. Both the membrane-bound isoforms and the soluble isoforms are immunotolerant, i.e. they inhibit NK cell-mediated and CTL-mediated cytolysis and also the alloproliferative T response; in addition, they induce apoptosis in CD8⁺ NK cells and T cells.

Thus, the HLA-G protein exercises its function locally, both when it is expressed at the cell surface and when it is secreted (remote action); it thus performs the immunosurveillance of the body (Teyssier Em. et al., Nat. Immunol., 1995, 14, 262-270).

Prior studies have shown that the expression of HLA-G molecules at the surface of target cells obtained by transfection with vectors comprising the genomic HLA-G DNA, potentially generating all the alternative transcripts, makes it possible to protect said target cells against the lytic activity of the NK cells of the decidual layer of the maternal endometrium (Chumbley G. et al., Cell Immunol., 1994, 155, 312-322; Deniz G. et al., J. Immunol., 1994, 152, 4255-4261).

The inventors, continuing their studies, have shown that certain solid tumors expressed the HLA-G antigen, that, depending on the tumor lines, the expression profile of the membrane-bound and soluble HLA-G isoforms was different, and that the presence of the membrane-bound HLA-G1-G4 forms protected the tumor cells against NK cell-induced lysis (application FR 2 775 294).

In addition, some of the inventors have shown the advantage of the soluble HLA-G forms in the treatment of inflammatory pathological skin conditions (PCT international application WO 00/78337).

The applicant has now found, surprisingly, other locations of the soluble HLA-G forms. In particular, these soluble HLA-G isoforms are present in the erythroid cells of placental vessels of the 1^{st} trimester of pregnancy, and also during early vascularization of the embryo and throughout erythropoiesis.

Thus, surprisingly, the inventors have shown that the soluble HLA-G isoforms also perform nonimmunological functions.

An aim of the invention is therefore to provide for novel applications of the soluble HLA-G isoforms, directly linked to their nonimmunological functions, in particular in blood circulation diseases such as anemia or ischemia.

A subject of the present invention is therefore the use of a composition comprising at least one soluble HLA-G isoform and at least one pharmaceutically acceptable vehicle, for preparing a medicinal product for use in the treatment of blood circulation diseases.

For the purpose of the present invention, the term “blood circulation diseases” is intended to mean both blood diseases and circulatory system (means of circulation) diseases.

According to an advantageous embodiment of said use, said disease is selected from the group consisting of anemias and ischemias.

In fact, the inventors have shown the advantage of the soluble HLA-G isoforms:

1. in the proliferation, differentiation and maturation of erythroblasts to reticulocytes.

The introduction of HLA-G can stimulate the maturation of circulating erythroblasts, which can be observed in certain circulatory system diseases (K. V. Wagner et al., Development, 2000, 127, 4949-4958; M. Ogawa et al., Blood, 1997, 50, 6, 1081-1092) and

2. in neovascularization (angiogenic process).

In accordance with the invention, said compositions are in one of the following forms:
liquid form, suitable for parenteral or oral administration,
solid form, suitable for parenteral administration after dissolution or suspension, or for oral administration.

The excipients suitable for these two forms are preferably: water, NaCl, dextrose, glycerol, ethanol or a combination of these products. Other substances, such as wetting agents, emulsifiers or buffers, can advantageously be added.

A subject of the present invention is also a method of detecting and, optionally, sorting cells with a nonimmunological function expressing a soluble HLA-G isoform (cells of the erythrocyte and endothelial lines), in a biological sample, which method is characterized in that it comprises at least the following steps:
(a) bringing the biological sample to be tested into contact with a panel of antibodies selected from the group consisting of antibodies directed against the following markers: soluble HLA-G isoform, CD71, CD34 and CD45, and
(b) detecting and, optionally, sorting cells corresponding to various stages of differentiation of the erythrocyte and endothelial lines, according to their profile of expression of the markers defined in (a).

According to an advantageous embodiment of said method, it comprises:
(a) bringing the biological sample to be tested into contact with a panel of antibodies selected from the group consisting of antibodies directed against: the following markers: soluble HLA-G isoform CD71, CD34 and CD45, and
(a′) selecting the cells expressing the soluble HLA-G isoform, and
(b) detecting the type of cell using the CD71 marker.

In fact, endothelial cells expressing HLA-G and cells of the erythroid line expressing HLA-G can be distinguished by the fact that only the latter express the CD71 marker.

In accordance with the invention, the biological sample is advantageously: a blood sample (detection of circulating cells of the erythroid line) or a bone marrow sample.

Definitions

Primitive hematopoiesis: in the yolk sack, in the first stages of development; short-term erythromyeloid reconstitution.

Production of hematopoietic stem cells (HSCs): the intraembryonic para-aortic splanchnopleura/aorta-gonad-mesonephros (P-Sp/AGM) region produces the HSCs, corresponding to the multipotent precursors capable of self-renewal which will subsequently colonize the fetal liver and the thymus.
Definitive hematopoiesis: begins in the bone marrow from the second trimester of intraembryonic life and continues throughout adult life.
Erythroid cells: the first hematopoietic cells to appear during intraembryonic life, which will give rise to the mature red blood cells.
Yolk sac: blood islets that appear in the yolk sac in the form of groups of cells between 15 and 24 days of embryonic development and which differentiate into endothelial cells from the outer cells of the clusters and into hematopoietic cells from the inner cells.
Hemangioblast: common precursor of endothelial cells and hematopoietic cells, present in the yolk sac.
Primary hematopoietic organs: embryonic liver, fetal bone marrow.
Budding vessels: in the chorionic villi and the juxta-allantoic portion of the yolk sac.

According to an advantageous embodiment of said method, prior to step a), the cells of said biological sample are permeabilized: this step allows the antibodies to effectively gain access to the markers to be detected that are secreted into the cytoplasm, before they pass into the general circulation (in the case of the soluble HLA-G isoforms).

Thus, distribution profiles characteristic of cells are obtained, as illustrated below:

| Cells/markers | CD71 | CD34 | HLA-G | CD45 |
|---|---|---|---|---|
| Erythroid line: (embryo, child | | | | |
| and adult individual) | | | | |
| HSC | − | + | − | + |
| BFU-E | + | + | + | − |
| CFU-E | + | − | + | − |
| Erythroblasts | + | − | + | − |
| Reticulocytes | + | − | + | − |
| Mature erythrocytes | − | − | − | − |
| Endothelial cells: | | | | |
| Endothelial stem cells | − | + | − | − |
| Budding vessel endothelial | − | + | + | − |
| cells (embryo) | | | | |
| Mature vessel endothelial cells | − | + | − | − |

In summary:
the soluble HLA-G isoform is expressed in all cells of the erythroid line and in all the hematopoietic organs, from embryo to adult, whereas it is not found in mature erythrocytes. In addition, no expression of a soluble HLA-G isoform is observed in CD34⁺ CD45⁺ hematopoietic stem cells (HSCs); this implies that the soluble HLA-G isoform has no action on multipotent stem cells.

It may also be noted that the soluble HLA-G isoform is produced, in vitro, during differentiation of the erythroid line, regardless of the stage of maturation. These results confirm that the soluble HLA-G isoform is involved in the proliferation and/or maturation of erythroid line precursors;
the soluble HLA-G isoform is expressed in endothelial cells at an early stage in the embryo, whereas it is not found in the endothelial cells of mature vessels. In particular, it is detected in the budding vessels of the chorionic villi and of the juxta-allantoic portion of the yolk sac.

Besides the above provisions, the invention also comprises other provisions that will emerge from the following description, which refers to examples of implementation of the present invention and also to the attached drawings, in which:

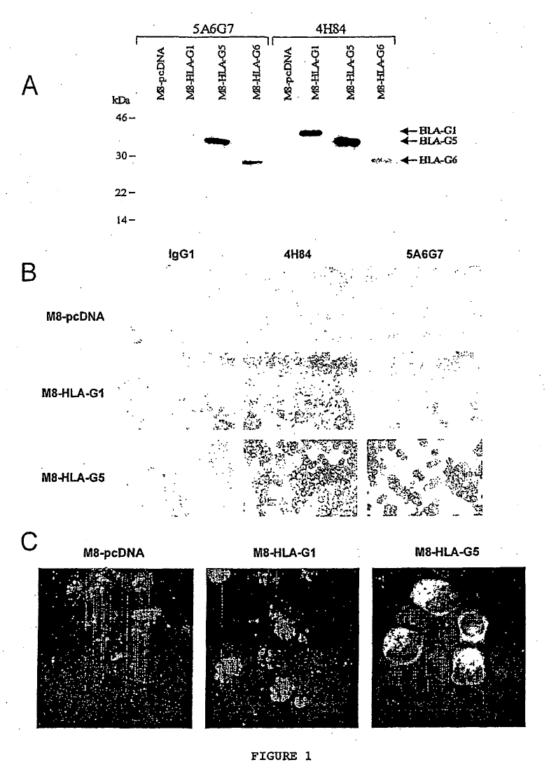

FIG. 1 illustrates the specificity of the 5A6G7 antibody with respect to the soluble HLA-G forms. FIG. 1A illustrates the results obtained by Western blotting. The specificity of the 5A6G7 antibody was tested on the M8-pcDNA, M8-HLA-G1, M8-HLA-G5 and M8-HLA-G6 lines; in parallel, the 4H84 antibody (McMaster et al., J. immunol., 1998 and Paul et al., Hum. Immunol., 2000) was used: the 4H84 antibody reveals 3 bands corresponding to HLA-G1, HLA-G5 and HLA-G6; the 5A6G7 antibody reveals 2 bands corresponding to HLA-G5 and HLA-G6. The M8-pcDNA line, which does not express HLA-G, is labeled neither with the 4H84 antibody nor with the 5A6G7 antibody. FIG. 1B shows the results obtained by immunocytochemistry. The specificity of the 5A6G7 antibody was tested on the M8-pcDNA, M8-HLA-G1 and M8-HLA-G5 lines, in parallel with the 4H84 antibody and an isotypic control. The positive labelings are characterized by a gray color. For each of the three lines, the antibody used for the labelings is indicated at the top. line 1: the M8-pcDNA line, which does not express HLA-G, is labeled neither with the 4H84 antibody nor with the 5A6G7 antibody. line 2: the M8-HLA-G1 line, which does not possess the epitope recognized by the 5A6G7 antibody, is only labeled with the 4H84 antibody. line 3: the M8-HLA-G5 line is labeled with the two antibodies 4H84 and 5A6G7 (magnification ×40). FIG. 1C illustrates the results obtained by immunofluorescence analyzed by confocal microscopy. The specificity of the 5A6G7 antibody was tested on the M8-pcDNA, M8-HLA-G1, M8-HLA-G5, FO and FO-HLA-G6 lines. As expected, the M8-HLA-G5 and FO-HLA-G6 cells are labeled with the 5A6G7B12 antibody, whereas the M8-pcDNA, M8-HLA-G1 and FO cells, which do not possess the epitope recognized by the 5A6G7 antibody, are not labeled. The 4H84 antibody is a reference antibody that specifically recognizes all the HLA-G isoforms;

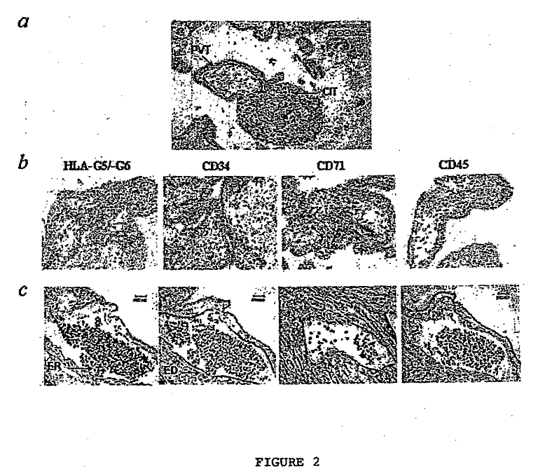

FIG. 2 illustrates the presence of the soluble HLA-G5 and G6 isoforms in the erythroblasts of trophoblasts from the first trimester of pregnancy. a: expression of the soluble isoforms by cell island trophoblasts or CIT, but not by PVT (perivillous trophoblasts), determined by immunohistochemical analysis using the 5A6G7 antibody on paraffin-embedded trophoblast sections; b: analysis of the expression of the following markers: HLA-G5/G6, CD34, CD71 and CD45 on developing vessels, present in the first trimester trophoblast, by immunohistochemistry; c: immunohistochemical staining of a differentiated vessel on sections of trophoblast of a 32-day embryo, with antibodies directed against HLA-G5/G6 (5A6G7), CD34 which labels the endothelial cells (ED) of the vessels, CD71 which identifies the erythroid cells (ER) and CD45 which targets both the myeloid cells and the lymphoid cells;

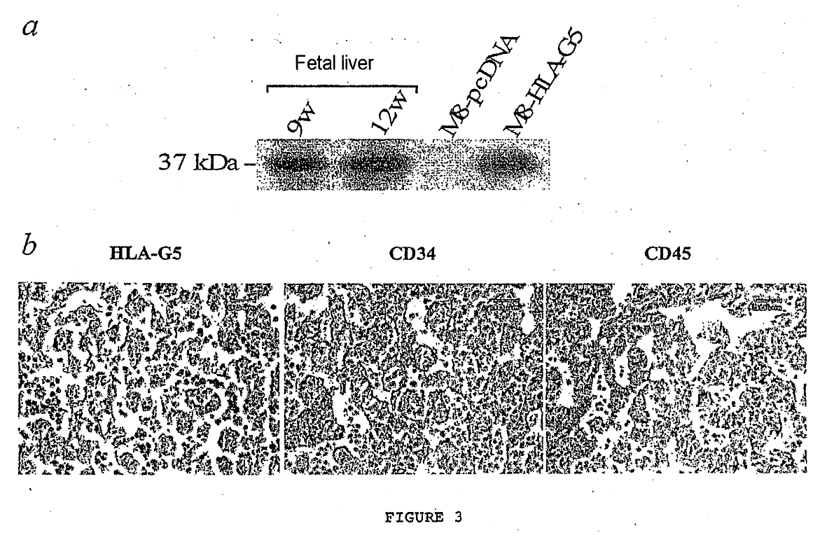

FIG. 3 illustrates the fact that HLA-G5 is present in the erythroid cells of fetal liver: a: identification of the soluble HLA-G5 isoform by running the total proteins extracted from two fetal livers (9 weeks and 12 weeks) on an SDS-PAGE gel, followed by immunoblotting with the 5A6G7 antibody. The M8 cells transfected with the HLA-G5 DNA (M8-HLA-G5) or with the control vector alone (M8-pcDNA) are used, respectively, as positive and negative controls; b: immunohistochemical analysis of a liver of a 32-day embryo with the antibodies directed against: HLA-G5, CD34 and CD45. HLA-G5 is detected in the erythroid cells that originate from the yolk sac and are present in the sinusoidal lumen (capillaries of the embryo). The endothelial cells are CD45⁺. A few cells associated with hepatocytes are CD34⁺ and CD45⁺ and can be considered as the first progenitor cells coming from the AGM;

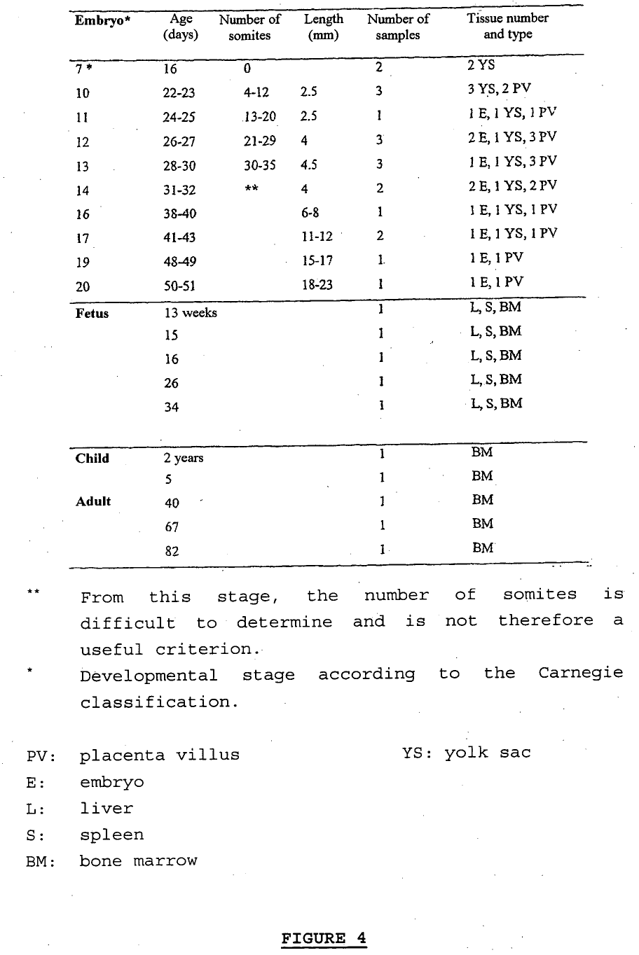

FIG. 4 illustrates the stage of the embryo, of the fetus, of the children and of the adults analyzed by immunohistochemistry.

### EXAMPLE 1

### Production of a Monoclonal Antibody Called 5A6G7, Directed Specifically Against the Soluble HLA-G Isoforms

1.1 Obtaining Ascites

The 5A6G7 monoclonal antibody is produced, using conventional protocols, from splenocytes of Balb/c mice immunized with a synthetic 21-mer peptide corresponding to the C-terminal portion encoded by intron 4 of the soluble HLA-G forms, the sequence of which is: SKEGDGGIMSVRESRSLSEDL (SEQ ID NO: 1), coupled to the ovalbumin (OVA) carrier protein.

1.2 Phase of Purifying the 5A6G7 Monoclonal Antibody

The 5A6G7 monoclonal antibody is purified from ascites using protein A-Sepharose affinity chromatography and can be used for the detection, titration and purification of the soluble HLA-G forms.

1.3 Test for Specificity with Respect to Soluble HLA-G5 and HLA-G6

The criteria required to obtain an antibody with good specificity are as follows:
detection of the 37 kDa HLA-G5 and 28 kDa HLA-G6 proteins but not of the other HLA-G isoforms and of the other class I HLA molecules present in the protein lysates from M8 cells (cells derived from the M8 melanoma cell line) transfected, respectively, with the vector pcDNA alone, or HLA-G1, -G2, -G3, -G4, -G5 or -G6 cDNAs; M8-pcDNA, MB-HLA-G1, M8-HLA-G5 and M8-HLA-G6 are cells derived from the M8 melanoma cell line transfected with the empty vector, HLA-G1, HLA-G5 or HLA-G6, respectively. FO and FO-HLA-G6 are cells derived from the FO melanoma cell line transfected with the empty vector or HLA-G6, respectively;
no detection of the HLA-A, -B, -C and -E proteins by Western blotting analysis of the protein lysates from human cell lines expressing different class I HLA types;
immunoprecipitation of the 37 kDa HLA-G5 protein from M8-HLA-G5 cell supernatant;
specific immunocytochemical staining of M8-HLA-G5 and M8-HLA-G6 cells but not of the other cells transfected with M8-pcDNA, -HLA-G1, -G3 and -G4, nor with peripheral blood mononuclear cells from several adult donors in good health expressing distinct class I HLA types;

immunohistochemical staining of paraffin-embedded trophoblast tissue sections but not of paraffin-embedded normal adult tissue sections (i.e. skin, liver, kidney, duodenum). This monoclonal antibody makes it possible notably to discriminate between the soluble HLA-G proteins generated by detachment of the membrane-bound HLA-G forms, which do not contain the epitope encoded by intron 4, and the HLA-G5/-G6 proteins produced from alternatively spliced mRNAs which contain intron 4.

### EXAMPLE 2

### Detection of the Soluble HLA-Gs in Circulating Cells of the Chorionic Vessels Originating from Placenta at the First Trimester Stage

The monoclonal antibody (5A6G7) described in example 1 made it possible to study the tissue distribution of the soluble HLA-G isoforms on tissue sections. In a preliminary analysis, placental tissue at the first trimester stage was analyzed.

2.1 Materials and Methods

2.1.1 Tissues

Human embryonic and fetal tissues were analyzed from archived slides originating from extra-uterine pregnancies, miscarriages and abortions from abnormal fetuses (essentially trisomies 21 and 18). The developmental stage of the embryo is estimated on the basis of several anatomical criteria according to the Carnegie classification (O'Rahilly et al., 1987). In total, 19 embryos, 6 fetuses and 5 adults were selected and a total of 56 tissues were analyzed by immunohistochemistry as illustrated in FIG. 4.

2.1.2 Monoclonal Antibodies

The 5A6G7 antibody was prepared as specified in example 1.
The 4H84 antibody is a reference antibody that recognizes all the HLA-G isoforms.
The monoclonal antibodies directed against the transferrin CD71 receptor (Novocastra Laboratories, UK) were used to identify erythroid cells.
The monoclonal antibodies directed against CD34 (Novocastra Laboratories) and CD45 (Dako, FR) make it possible to detect hematopoietic progenitor cells, whereas only the monoclonal antibody directed against CD34 makes it possible to recognize endothelial progenitor cells.
   2.1.3. Erythroid Cell Cultures

Cultures of erythroid cells (1.10⁵ umbilical cord blood cells or bone marrow cells) were plated out on a semi-solid medium containing methylcellulose, 10% of LCM (lymphocyte conditioned medium) (Stem cell, Vancouver) and 2 U/ml of EPO (Roche, FR). These cultures are incubated at 37° C. in an atmosphere containing 5% CO₂, for 14 days.

Two-phase liquid culture is used and is defined as follows. Briefly, the cells (of the umbilical cord, for example) are isolated by centrifugation on a Ficoll 1077 (SIGMA) gradient, and placed in culture at a density of 10⁶ cells/ml in alpha minimal essential medium (α-MEM, Sigma) supplemented with 10% of fetal calf serum (FCS, Sigma), 1 μg/ml of cyclosporin A (Novartis) and 10% of medium recovered from HLA-G-negative 5637 bladder carcinoma cell lines (ATCC no. HTB-9). The cultures are incubated for 7 days at 37° C. under an atmosphere containing 5% CO₂, in air with 98% humidity. After this phase I culture, the nonadherent cells are recovered, washed, and seeded at a density of 0.4×10⁶ cells/ml in α-MEM, 30% FCS, 1% bovine serum albumin, 10⁻⁵M of β-mercaptoethanol, 15 mM of L-glutamine, 10⁻⁶ M of dexamethasone and 1 U/ml of recombinant erythropoietin (Epo, Roche) for 5-6 days (called phase II culture).

2.1.4. Immunohistochemical Analysis

Deparaffinized tissue sections are subjected to a high-temperature epitope recovery treatment in 10 mM sodium citrate buffer (pH 6.0) using a commercial microwave, in order to optimize the immunoreactivity. Tissue sections were permeabilized using 1×PBS with 0.1% saponin and 10 mM Hepes buffer. Endogenous peroxidase activity is inhibited by treating the sections for 5 minutes at ambient temperature with 3% of hydrogen peroxide in water. Nonspecific binding is prevented by adding 50 mM Tris, 3% BSA (Sigma) and 40% human serum for 20 minutes, before staining with the primary antibody for 30 minutes at ambient temperature. Expression of the HLA-G protein is then evaluated on a series of tissue sections or cells using the UltraTech HRP universal biotin detection system (Immunotech, Coulter, France), according to the supplier's instructions. Immunocytochemical analysis of HLA-G expression by BFU-E cells is carried out on cytospun BFU-E cells which have been recovered from the cultures in semi-solid medium and washed in PBS, before the spinning.

2.2 Results

As shown in FIG. 2a, the HLA-G5 and HLA-G6 proteins are localized in the extravillous trophoblast cells. In addition, and surprisingly, these soluble HLA-G molecules were detected, by staining with the 5A6G7 monoclonal antibodies, either in cells in close contact with budding vessels associated with cytotrophoblasts, or in cells devoid of villous axes, or in cells located in the lumen of mature vessels (FIGS. 2b and 2c). This new cellular localization of HLA-Gs is confirmed using another anti-HLA-G antibody, the reference 87G monoclonal antibody.

In order to identify more precisely these cells expressing the HLA-G protein, which morphologically resemble erythroblasts, an immunohistochemical analysis was carried out on a series of paraffin-embedded trophoblast sections originating from 32-day embryos, by detection of the following markers:
the CD71 transferrin receptor: expressed from erythroids (BFU-E, burst-forming unit) to reticulocytes,
CD34: expressed both by endothelial and hematopoietic progenitor cells,
CD45: expressed from hematopoietic progenitor cells up to mature cells of the lymphoid and myeloid lines,
the soluble HLA-G5 and HLA-G6 proteins.

The results show that the soluble isoforms are expressed by the entire subpopulation of CD71⁺ erythroid cells, whereas few HLA-G⁺ cells coexpress the CD34 stem cell marker (FIGS. 2b and 2c).

Conversely, the endothelial cells of mature chorionic vessels are CD34⁺, HLA-G⁻, CD71⁻ and CD45⁻ (FIG. 2c).

These results make it possible effectively to conclude that the soluble HLA-G isoforms are present in hematopoietic cells belonging to the erythropoietic line.

### EXAMPLE 3

### Identification of the Soluble HLA-G5 Isoform in Fetal Erythroblasts

In order to characterize the soluble HLA-G isoform present in fetal erythroid cells, the organ in which erythroblasts are the main constituents, namely the liver, which is the main producer of red blood cells in the fetus, was used.

3.1 Materials and Methods

3.1.1 Tissues and Monoclonal Antibodies

The proteins are extracted from the liver obtained from 2 distinct embryos, respectively at 9 and 12 weeks. The tissues on which the analyses are carried out are those described in example 2.

The 5A6G7 monoclonal antibody described in example 1 was used for the immunohistochemical analysis.

3.1.2 Western Blotting Analysis

9- and 12-week fetal livers were ground and lysed in 50 mM of Tris-HCl, pH 7.4, 150 mM of NaCl, 1% Nonidet P-40 (Sigma) containing protease inhibitors and cold buffer for 1 hour. The lysates of cells transfected with M8-HLA-G5 and M8-pcDNA were used as, respectively, positive and negative controls with respect to HLA-G5. After centrifugation at 15 000 g at 4° C. for 30 minutes, 6× Laemmli buffer was added to the supernatants. The samples were heated for 10 minutes at 95° C. before being loaded onto a 12% SDS-PAGE gel. The proteins were then electro-blotted onto a nitrocellulose membrane (Hybond-C extra). The membrane was then treated against nonspecific binding with 5% dried skimmed milk in PBS containing 0.2% of Tween 20 (PBST) overnight at 4° C. After washing in PBST, the membrane was incubated with HRP-conjugated anti-mouse antibodies for 30 minutes at ambient temperature and washed in PBST. The HLA-G proteins were detected by chemiluminescence (ECL Plus Kit) (Menier et al., Hum. Immunol., 2003, 64, 315-326).

3.2 Results

By Western blotting using the 5A6G7 monoclonal antibody, a protein of 37 kDa, which corresponds to the molecular weight of the HLA-G5 heavy chain (FIG. 3), is identified. The M8-pcDNA and M8-HLA-G5 transfectants are used, respectively, as positive and negative controls with respect to HLA-G5. This result shows that the soluble HLA-G isoform detected in the fetal erythroblasts is the HLA-G5 isoform. In agreement with this result, an immunohistochemical analysis made it possible to detect many HLA-G⁺ cells (incubation with the 5A6G7 monoclonal antibody) in paraffin-embedded fetal liver tissue, as detailed in the following example.

### EXAMPLE 4

### Distribution of the Soluble HLA-G5 Isoform in All the Embryonic and Fetal Hematopoietic Organs

The distribution of the soluble HLA-G5 isoform in all the embryonic and fetal hematopoietic organs was analyzed by carrying out immunohistochemical experiments on paraffin-embedded fetal tissue and embryo sections using the monoclonal antibodies directed against HLA-G5, CD71, CD34 or CD45.

4.1 Materials and Methods

The tissues analyzed come from embryonic and fetal hematopoietic organs as described in example 2.

The monoclonal antibodies directed against HLA-G5 (5A6G7) and the markers CD71, CD34 and CD45 of example 1 were used under the conditions specified in example 2.

4.2 Results

Colocalization of HLA-G and of the CD71 receptor is observed in all the embryonic and fetal hematopoietic organs: yolk sac, para-aortic splanchnopleura/aorta-gonad-mesonephros (P-Sp/AGM), liver, spleen and bone marrow; these results confirm that the HLA-G⁺ cells are erythroblasts.

| Erythroid cells | CD71 | CD34 | HLA-G | CD45 |
|---|---|---|---|---|
| Yolk sac | + | − | + | − |
| pSp/AGM | + | − | + | − |
| fetal liver | + | − | + | − |
| spleen | + | − | + | − |
| bone marrow | + | − | + | − |

These results show that the HLA-G⁺ cells also express the CD71 receptor, and this colocalization exists throughout embryonic and fetal development: in the yolk sac, in the 16-day embryo, in all the hematopoietic organs of a 32-day embryo, in the 12-, 34- and 36-week liver, in the 12-, 14- and 16-week bone marrow, and also in the bone marrow in adults. They are therefore clearly a marker for the erythroid line.

These results therefore illustrate the role of the soluble HLA-G isoform in erythropoiesis as a marker in the differentiation of the erythroid line.

Moreover, these results show that no CD34+ hematopoietic stem cell is present in the yolk sac of a 16-day embryo, unlike the CD71⁺ erythroid stem cells.

In fact, the CD34⁺, CD45⁺ hematopoietic stem cells are all HLA-G⁻.

This shows that HLA-G is not involved on multipotent stem cells.

### EXAMPLE 5

### Umbilical Cord Blood Erythroblasts Express HLA-G5

In order to determine whether the HLA-G5 isoform is produced directly by erythroblasts or else attached to the cell surface of the erythroblast via specific HLA-G receptors, after it has been produced by other cells, umbilical cord blood was chosen as a source of erythroblasts.

5.1 Materials and Methods

5.1.1 Tissues

Units of umbilical cord blood (p/CB) were obtained during normal term deliveries, after informed consent from the mothers, at the obstetrics unit of the Robert Debre Hospital in Paris (France).

5.1.2 Erythroid Burst Forming Cell (BFU-E) Trials in Semi-Solid Medium

see Example 2, erythroid cell culture chapter.

5.1.3 Maturation of Progenitor Cells in 2-Phase Liquid Culture

See example 2, erythroid cell culture chapter.

The concentration of HLA-G5 in the supernatants is evaluated by means of an ELISA, which specifically measures HLA-G5/G6.

5.1.4 ELISA Assay

The HLA-G5 concentrations are measured in two-phase liquid culture supernatants at the end of phases I and II (“sandwich”-type method). 96-well microtitration plates (Corning Costar, France) are coated with 5A6G7 monoclonal antibodies. After 5 washes in phosphate buffered saline (PBS) containing 0.2% of Tween 20 and 0.1% of bovine serum albumin (BSA, Sigma), the plates are saturated with PBS containing 1% of BSA and 0.1% of Tween 20, for 2 hours at 37° C. After 5 washes in PBS with 0.2% of Tween 20, 100 μl of sample are added to each well and assayed in duplicate. After incubation for 1 hour at 37° C., the plates are washed 5 times in PBST (Phosphate Buffered Saline Tween). The detection antibody, a biotinylated monoclonal antibody W6/32 (Leinco Technologies Inc., Ballwin) which recognizes a monomorphic determinant of the class I HLA heavy chains associated with β2-microglobulin, diluted beforehand to 1/250, was incubated for 1 hour at 37° C. After washing in PBST, the visualization is carried out by incubation in AMDEX™ streptavidin horseradish peroxidase conjugate (Amersham) for 1 hour at 37° C., and then with TMB substrate (3,3′, 5,5′-tetramethylbenzidine, Sigma) at ambient temperature. The reaction is stopped by adding 1N HCl. The optical densities are measured at 450 nm. Standard curves are produced using serial dilutions of purified recombinant soluble HLA-G5. The HLA-G5 concentration is determined from the optical density value according to the standard curve. The results are expressed as the means of the duplicate. The limit of detection by ELISA assay is 5 ng/ml.

5.2 Results

Whatever the state of the culture, i.e. from the most primitive cell (BFU-E) to the most differentiated erythroid cell (reticulocyte), the HLA-G5 concentration is between 25.5 ng/ml and 44.3 ng/ml. An immunocytochemical analysis of the corresponding cells confirms that the two-phase liquid culture makes it possible to differentiate erythroid cells which are positive for CD71 and coexpress CD36 (thrombospondin receptor and marker for identifying the CFU-E and erythroblast stages of the erythroid line) at D=7 of the second culture phase. According to these results, it may be concluded therefrom that the HLA-G5 molecules detected by ELISA in culture supernatants were indeed produced by the erythroid cells in the course of their differentiation.

### EXAMPLE 6

### Erythroblasts of Adult Bone Marrow Expressing HLA-G5

The maintenance of expression of the HLA-G5 isoform in the erythropoeitic line throughout the lifetime in adults was studied. Given that hematopoiesis is located in the bone marrow in adults, the BFU-E cells were collected from semi-solid culture of bone marrow hematopoietic progenitor cells (see example 2).

6.1 Materials and methods

6.1.1 Monoclonal Antibodies

The following monoclonal antibodies were used to detect HLA-Gs:
4H84 is directed against peptide 61-83 of the α1 domain of HLA-G and recognizes the free HLA-G heavy chain;
MEM-G/09 (Exbio, Prague, Czech Republic).

All these antibodies react with a correctly folded β2m-associated HLA-G molecule.

6.1.3 Methods

The technique of culturing BFU-E cells in semi-solid medium and the immunochemical analysis were used as described in example 2.

6.2 Results

The soluble HLA-G5 isoform is detected in the BFU-E cells by immunocytochemical analysis using several anti-HLA-G monoclonal antibodies, such as 5A6G7, MEM-G/9 and 4H84. In addition, in an immunochemical analysis on paraffin-embedded bone sections, HLA-G5 is then localized in the erythroblasts from adult bone marrow.

### EXAMPLE 7

### Expression of the Soluble HLA-G Isoform by Embryonic Endothelial Cells

The soluble HLA-G isoform is also identified in the endothelial cells in budding vessels at two sites: in the mesenchymal core of chorionic villi and in the juxta-allantoic portion of the yolk sac of early embryos.

In each case, the HLA-G isoform colocalizes with the CD34 marker.

The presence of the soluble HLA-G isoform in the endothelial cells of the budding vessels of chorionic villi and of the juxa-allantoic portion of the yolk sac shows its involvement in the angiogenic process.

### EXAMPLE 8

### Effect of HLA-G5 on Erythroid Differentiation

The effect of HLA-G5 on erythroid differentiation was analyzed on an erythroleukemia line (K562, ATCC) expressing or not expressing HLA-G5, namely: a K562 line transfected with an HLA-G5 expression plasmid and secreting HLA-G5 (K562-pcDNA-HLA-G5) and, as a control, a K562 line transfected with the empty vector (K562-pcDNA).

The expression of the differentiation markers glycophorin A (GPA) and CD36, which appear at the final stages of erythroid differentiation (erythroblast, reticulocyte, mature red blood cell), was analyzed by flow cytometry using commercial antibodies directed against these markers. The results are given below:

| Cells | GPA | CD36 |
|---|---|---|
| K562-pcDNA-HLA-G5 | 40% (16) * | 77% (15) |
| K562-pcDNA | 22% (8.7) | 55% (10) |

* the values between parentheses correspond to the mean fluorescence intensity.

The immunolabeling results show that the HLA-G5-secreting line consists of a larger number of mature erythroid cells, which express a greater amount of erythrocyte differentiation markers.

The effect of HLA-G5 on erythroid differentiation is confirmed by coculture experiments between the two lines, in order to verify that the HLA-G5 form secreted by K562-pcDNA-HLA-G5 induces differentiation of the K562-pcDNA line.

As emerges from the above, the invention is in no way limited to those of its methods of implementation, execution and application which have just been described more explicitly; on the contrary, it encompasses all the variants thereof that may occur to those skilled in the art, without departing from the context or the scope of the present invention.

1. A method of treating at least one blood circulation disease in a patient in need thereof comprising administering at least one soluble HLA-G isoform to the patient in an amount sufficient to treat the at least one blood circulation disease.

2. The method of claim 1, wherein the at least one blood circulation disease is selected from the group consisting of anemias and ischemias.

3. A composition comprising at least one soluble HLA-G isoform and at least one pharmaceutically acceptable vehicle.

4. The composition of claim 3, wherein the composition is formulated as a liquid.

5. The composition of claim 3, wherein the composition is formulated as a solid.

6. A method of detecting and, optionally, sorting cells of the erythrocyte lines, the endothelial lines, or combinations thereof, according to their state of differentiation, comprising:
a) bringing a biological sample to be tested into contact with antibodies directed against the following markers: soluble HLA-G isoform, CD71, CD34 and CD45, and
b) detecting and, optionally, sorting cells corresponding to various stages of differentiation of the erythrocyte lines, the endothelial lines, or a combination thereof, according to their profile of expression of the markers defined in a).

7. The method as claimed in claim 6, further comprising
a′) selecting the cells expressing the soluble HLA-G isoform, and
c) detecting the type of cell using the CD71 marker.

8. The method of claim 6, wherein the biological sample is selected from the group consisting of a blood sample and a bone marrow sample.

9. The method of claim 6, wherein prior to a), the cells of said biological sample are permeabilized.

10. The method of claim 7, wherein the biological sample is selected from the group consisting of a blood sample and a bone marrow sample.

11. The method of claim 7, wherein prior to a), the cells of said biological sample are permeabilized.

12. The method of claim 8, wherein prior to a), the cells of said biological sample are permeabilized.
